(19) United States
(12) Patent Application Publication
(10) Pub. No.: US 2010/0312327 A1
(43) Pub. Date: Dec. 9, 2010

(54) MEDICAL DEVICE OF TERNARY ALLOY OF MOLYBDENUM AND RHENIUM

(75) Inventors: Pamela A. KRAMER-BROWN, San Jose, CA (US); John F. BOYLAN, Murrieta, CA (US); Randolf VON OEPEN, Los Altos, CA (US)

Correspondence Address: FULWIDER PATTON, LLP (ABBOTT), 6060 CENTER DRIVE, 10TH FLOOR, LOS ANGELES, CA, 90045, US

(73) Assignee: ABBOT CARDIOVASCULAR SYSTEMS, INC., Santa Clara, CA (US)

(21) Appl. No.: 12/479,440

(22) Filed: Jun. 5, 2009

(51) Int. Cl. A61F 2/82; C22C 27/04; C22C 30/00

(52) U.S. Cl. 623/1.15; 420/429; 420/580

(57) ABSTRACT

A medical device such as, for example, an implantable expandable stent is constructed of a ternary alloy of molybdenum, rhenium, and a third metal. In a preferred embodiment, the third metal is a refractory metal selected to improve the ductility of the alloy. The alloy may further be advantageously constructed to have a crystal structure selected from HCP, BCC, FCC, and tetragonal to further optimize the physical characteristics of the medical device.

### BACKGROUND

Medical treatment of various illnesses or diseases commonly includes the use of one or more medical devices. When a body lumen requires repair, one type of medical device commonly employed is an expandable stent. Stents may be used to open an obstructed or partially obstructed body lumen such as an artery or other blood vessel. When a stent is used in a blood vessel, a delivery system is used to place and then expand the stent to open the damaged vessel and facilitate improved blood flow. The procedure of opening a blocked or partially blocked body passageway commonly includes the use of one or more stents in combination with other medical devices such as, but not limited to, an introducer sheath, a guiding catheter, a guide wire, an angioplasty balloon, etc.

Various physical attributes of a stent can contribute directly to the success rate of the device. These physical attributes include radiopacity, hoop strength, radial force, thickness of the metal, dimensions of the metal, and the like. Materials such as cobalt alloys and stainless steels are commonly used to form stents, guidewires, and other medical devices. These materials are selected on the basis of their known history of safety, effectiveness and biocompatibility. These materials however have limited physical performance characteristics as they relate to size, strength, weight, workability, bendability, biostability and radiopacity.

Refractory metals such as molybdenum (Mo), niobium (Nb), tungsten (W), rhenium (Re), and tantalum (Ta) are generally characterized as having high melting temperatures due to their relatively strong interatomic bonds. In addition, these metals tend to exhibit high strength, large elastic moduli, and extreme hardness. Some of these alloys also exhibit very good corrosion resistance, which is an important consideration in the manufacture of medical devices. Also, they tend to have good radiopacity, another desirable trait of medical devices.

Paradoxically, the same properties (hardness, melting temperature) that make these alloys a good candidate for constructing medical devices such as intraluminal stents also make them difficult to process into those same medical devices. As manufacturing technologies have advanced, however, these materials become more practical for medical device applications. Refractory metals can also exhibit low ductility, so alloying may be used to increase the ductility for use in certain medical devices such as stents and the like. However, most alloys to date have focused on binary alloys, which have not been shown to be ideal for these applications.

There is a need in the art for improved medical devices that have favorable material characteristics in relation to medical devices presently known and used in the art. The present invention is generally directed to a medical device such as, but not limited to, a stent that is at least partially formed of a novel ternary metal alloy that improves the physical properties of the medical, device thereby improving the success rate of such medical device.

### SUMMARY OF THE INVENTION

The present invention provides for a medical device fabricated at least in part from a ternary alloy comprising molybdenum and rhenium. In one embodiment of this invention, molybdenum and rhenium are used as two of the alloy constituents in a ternary alloy comprising a refractory metal. These constituents provide for a material that has high strength and excellent radiopacity for use in a medical implant such as a stent. Further, in one preferred embodiment the ternary alloy of molybdenum and rhenium further includes a third metal constituent whose primary purpose is to improve the ductility of the alloy, making it better suited for use in a balloon expandable stent implant.

In addition to variations of the constituents and compositions that can be used to create a suitable ternary alloy for use in a stent in accordance with this invention, the metallic crystal structure may be varied as well. For example, a number of alloys may be formed in the hexagonal close-packed (HCP), body-centered cubic (BCC), face-centered cubic (FCC), and tetragonal crystal structure. Each of these crystal structures are known to provide specific advantages, and the ternary alloys described by this invention may be formed with any of these crystal structures to achieve these advantages.

The use of ternary alloys of the present invention provide for medical implants with particularly useful characteristics. For example, when used to construct an intraluminal stent implant, the ternary alloys of the present invention yield stents with thin struts and adequate radiopacity. The stents using the ternary alloys exhibit improved ductility over existing binary alloy refractory metal stents.

### BRIEF DESCRIPTION OF THE DRAWINGS

FIG. 1 illustrates a catheter and balloon delivery system for delivering a stent of the present invention, the catheter and balloon delivery system is shown inserted in a body lumen;

FIG. 2 illustrates the catheter and balloon delivery system of FIG. 1 where the balloon is expanded to deploy the stent within the body lumen; and

FIG. 3 illustrates the stent of the present invention deployed in the body lumen with the catheter and balloon delivery system withdrawn.

### DETAILED DESCRIPTION OF THE PREFERRED EMBODIMENTS

The use of stents in medical procedures is well known in the art. U.S. Pat. No. 7,331,987 discusses the use of stents in such procedures, and the contents of the '987 patent is fully incorporated herein by reference. The present invention when employed as a stent improves on existing stents by providing a unique ternary alloy material that improves the physical properties of the stent.

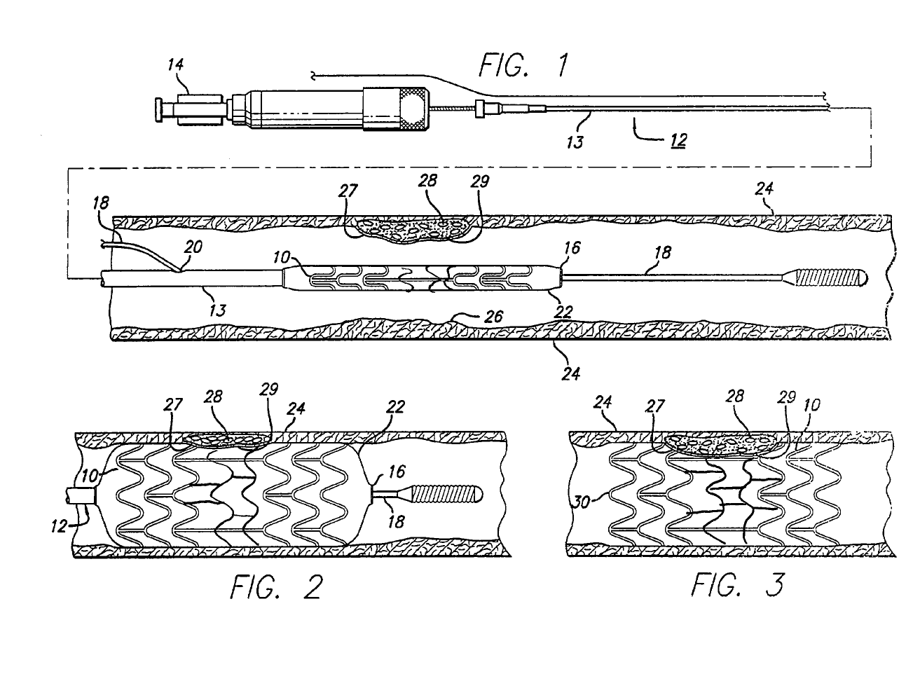

FIG. 1 depicts a stent 10 comprised of the ternary alloy of the present invention mounted on a catheter assembly 12 which is used to deliver the stent and implant it in a body lumen, such as a coronary artery, peripheral artery, or other vessel or lumen within the body. The catheter assembly includes a catheter shaft 13 which has a proximal end 14 and a distal end 16. The catheter assembly is configured to advance through the patient's vascular system by advancing over a guide wire by any of the well known methods of an over the wire system (not shown) or a well known rapid exchange catheter system, such as the one shown in FIG. 1.

The catheter assembly 12, as depicted in FIG. 1, is of the well known rapid exchange type which includes an RX port 20 where the guide wire 18 will exit the catheter. The distal end of the guide wire 18 exits the catheter distal end 16 so that the catheter advances along the guide wire on a section of the catheter between the RX port 20 and the catheter distal end 16. As is known in the art, the guide wire lumen which receives the guide wire is sized for receiving various diameter guide wires to suit a particular application. The stent is mounted on the expandable member 22 (balloon) and is crimped tightly thereon so that the stent and expandable member present a low profile diameter for delivery through the arteries.

As shown in FIG. 1, a partial cross-section of an artery 24 is shown with a small amount of plaque 26 that has been previously treated by an angioplasty or other repair procedure. The stent 10 of the present invention is used to repair a diseased or damaged arterial wall which may include the plaque 26 as shown in FIG. 1, or vulnerable plaque 27 which is commonly found in the coronary arteries, peripheral arteries and other vessels. Vulnerable plaque consists of a thrombogenic lipid 28 that is covered by a thin fibrous cap 29. The stent of the invention is configured to repair the vessel having both plaque and vulnerable plaque.

In a typical procedure to implant the stent 10, the guide wire 18 is advanced through the patient's vascular system by well known methods so that the distal end of the guide wire is advanced past the plaque or diseased area 26. Prior to implanting the stent, the cardiologist may wish to perform an angioplasty procedure or other procedure (i.e., atherectomy) in order to open the vessel and remodel the diseased area. Thereafter, the stent delivery catheter assembly 12 is advanced over the guide wire so that the stent is positioned in the target area. The expandable member or balloon 22 is inflated by well known means so that it expands radially outwardly and in turn expands the stent radially outwardly until the stent is apposed to the vessel wall. The expandable member is then deflated and the catheter withdrawn from the patient's vascular system. The guide wire is typically left in the lumen for post-dilatation procedures, if any, and subsequently is withdrawn from the patient's vascular system. As depicted in FIG. 2, the balloon is fully inflated with the stent expanded and pressed against the vessel wall, and in FIG. 3, the implanted stent remains in the vessel after the balloon has been deflated and the catheter assembly and guide wire have been withdrawn from the patient.

The stent 10 serves to hold open the artery after the catheter is withdrawn, as illustrated by FIG. 3. Due to the formation of the stent from an elongated tubular member, the undulating components of the stent are relatively flat in transverse cross-section, so that when the stent is expanded, it is pressed into the wall of the artery and as a result does not interfere with the blood flow through the artery. The stent is pressed into the wall of the artery and will eventually be covered with smooth muscle cell growth which further minimizes blood flow interference. The undulating portion of the stent provides good tacking characteristics to prevent stent movement within the artery.

The present invention provides for a stent or other medical device to be fabricated at least in part from a ternary alloy comprising molybdenum and rhenium. Binary alloys of molybdenum with rhenium have been proposed as constituents for making stents, as in U.S. Pat. No. 7,452,502, the contents of which are incorporated herein by reference. As discussed above, these binary alloys have low ductility and other characteristics that make their use in stents and other medical devices less than ideal. The present invention alloys the molybdenum and rhenium with a third constituent to produce a ternary alloy. For the medical devices constructed of the ternary alloy of molybdenum, rhenium, and a third constituent, the shortcomings of the workability and strength of the binary alloys are overcome, providing thinner, stronger stents with improved radiopacity and workability. A table is shown below in accordance with this invention showing selected ternary alloys and their typical crystal structure, although the crystal structures are not limited to those shown:

TABLE 1
Sampling of Potential Ternary Alloys with Refractory Metal Constituents

| Hexagonal Close | Body Center | Face Center | |
|---|---|---|---|
| Pack | Cubic | Cubic | Tetragonal |
| MoRuRe | MoNbRe | MoPtRe | MoRuRe |
| MoOsRe | MoVRe | MoIrRe | MoOsRe |
| MoNbRe | MoCrRe | MoPdRe | |
| MoVRe | MoTaRe | MoRhRe | |
| MoCrRe | MoWRe | MoAuRe | |
| MoTaRe | MoRuRe | MoAgRe | |
| MoWRe | MoOsRe | | |
| MoPtRe | MoPtRe | | |
| MoIrRe | MoIrRe | | |
| MoPdRe | MoPdRe | | |
| MoRhRe | MoRhRe | | |
| MoAuRe | MoAuRe | | |
| MoAgRe | MoAgRe | | |

Compositions of the ternary alloys shown above may be varied to achieve the desired material properties. For example, the ternary alloys of Table 1 exhibit beneficial radiopacity, high corrosion resistance, good workability, and high strength. The third metal in the alloy is preferably selected to improve the ductility of the alloy, making it better suitable for a medical device such as a balloon expandable stent implant.

As set forth in Table 1, in addition to the variations in the constituents and compositions that can be used to create a suitable ternary alloy for use in a stent, the metallic crystal structure may be varied as well. For example, a number of alloys may be formed in the hexagonal close-pack (HCP) crystal structure, the body-centered cubic (BCC) crystal structure, the face-centered cubic (FCC) crystal structure, and tetragonal crystal structure. Each of these crystal structures provide specific advantages, and so the ternary alloys described herein are also selected to utilize those known advantages. In a preferred embodiment, molybdenum composition is expected to be in the range of about 20-60%.

Various phase diagrams for the constituents listed in table 1 can be accessed at the ASM International web site, www.asminternational.org, along with the crystal data and melting temperatures for each combination, the contents of which are incorporated herein by reference.

Stents may be formed using well known manufacturing processes using tubing that is produced form the ternary alloys shown above. For that matter, tubing formed from any ternary alloy comprising at least one refractory metal constituent may be used to produce a stent with well known processes.

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall therebetween.

We claim:

1. A medical device formed of a metal alloy of molybdenum, rhenium, and a third constituent metal to form a ternary alloy, the third constituent metal contributing to an improved ductility of the metal alloy.

2. The medical device of claim 1, wherein the third constituent metal is a refractory metal.

3. The medical device of claim 2 wherein the third constituent is ruthenium.

4. The medical device of claim 3 wherein a crystal structure of the metal alloy is hexagonal close pack (HCP).

5. The medical device of claim 3 wherein a crystal structure of the metal alloy is tetragonal.

6. The medical device of claim 3 wherein a crystal structure of the metal alloy is body center cubic (BCC).

7. The medical device of claim 2 wherein the third constituent is Osmium.

8. The medical device of claim 7 wherein a crystal structure of the metal alloy is hexagonal close pack (HCP).

9. The medical device of claim 7 wherein a crystal structure of the metal alloy is tetragonal.

10. The medical device of claim 7 wherein a crystal structure of the metal alloy is body center cubic (BCC).

11. The medical device of claim 2 wherein the third constituent is niobium.

12. The medical device of claim 11 wherein a crystal structure of the metal alloy is body centered cubic (BCC).

13. The medical device of claim 11 wherein a crystal structure of the metal alloy is hexagonal close pack (HCP).

14. The medical device of claim 2 wherein the third constituent is vanadium.

15. The medical device of claim 14 wherein a crystal structure of the metal alloy is body centered cubic (BCC).

16. The medical device of claim 14 wherein a crystal structure of the metal alloy is hexagonal close pack (HCP).

17. The medical device of claim 2 wherein the third constituent is chromium.

18. The medical device of claim 17 wherein a crystal structure of the metal alloy is body centered cubic (BCC).

19. The medical device of claim 17 wherein a crystal structure of the metal alloy is hexagonal close pack (HCP).

20. The medical device of claim 2 wherein the third constituent is tantalum.

21. The medical device of claim 20 wherein a crystal structure of the metal alloy is body centered cubic (BCC).

22. The medical device of claim 20 wherein a crystal structure of the metal alloy is hexagonal close pack (HCP).

23. The medical device of claim 2 wherein the third constituent is tungsten.

24. The medical device of claim 23 wherein a crystal structure of the metal alloy is body centered cubic (BCC).

25. The medical device of claim 23 wherein a crystal structure of the metal alloy is hexagonal close pack (HCP).

26. The medical device of claim 2 wherein the third constituent is platinum.

27. The medical device of claim 26 wherein a crystal structure of the metal alloy is face centered cubic (FCC).

28. The medical device of claim 26 wherein a crystal structure of the metal alloy is hexagonal close pack (HCP).

29. The medical device of claim 26 wherein a crystal structure of the metal alloy is body centered cubic (BCC).

30. The medical device of claim 2 wherein the third constituent is iridium.

31. The medical device of claim 30 wherein a crystal structure of the metal alloy is face centered cubic (FCC).

32. The medical device of claim 30 wherein a crystal structure of the metal alloy is hexagonal close pack (HCP).

33. The medical device of claim 30 wherein a crystal structure of the metal alloy is body centered cubic (BCC).

34. The medical device of claim 2 wherein the third constituent is palladium.

35. The medical device of claim 34 wherein a crystal structure of the metal alloy is face centered cubic (FCC).

36. The medical device of claim 34 wherein a crystal structure of the metal alloy is hexagonal close pack (HCP).

37. The medical device of claim 34 wherein a crystal structure of the metal alloy is body centered cubic (BCC).

38. The medical device of claim 2 wherein the third constituent is rhodium.

39. The medical device of claim 38 wherein a crystal structure of the metal alloy is face centered cubic (FCC).

40. The medical device of claim 38 wherein a crystal structure of the metal alloy is hexagonal close pack (HCP).

41. The medical device of claim 38 wherein a crystal structure of the metal alloy is body centered cubic (BCC).

42. The medical device of claim 2 wherein the third constituent is silver.

43. The medical device of claim 42 wherein a crystal structure of the metal alloy is face centered cubic (FCC).

44. The medical device of claim 42 wherein a crystal structure of the metal alloy is hexagonal close pack (HCP).

45. The medical device of claim 42 wherein a crystal structure of the metal alloy is body centered cubic (BCC).

46. The medical device of claim 2 wherein the third constituent is gold.

47. The medical device of claim 46 wherein a crystal structure of the metal alloy is face centered cubic (FCC).

48. The medical device of claim 46 wherein a crystal structure of the metal alloy is hexagonal close pack (HCP).

49. The medical device of claim 46 wherein a crystal structure of the metal alloy is body centered cubic (BCC).

50. The medical device of claim 1 wherein the molybdenum composition is in a range of about twenty to sixty percent by weight.

51. The medical device of claim 1 wherein the medical device is a stent implant.
